(19) United States
(12) Patent Application Publication
(10) Pub. No.: US 2024/0293490 A1
(43) Pub. Date: Sep. 5, 2024

(54) CHRYSANTHEMUM ZAWADSKII EXTRACT AND PREPARING METHOD THEREOF

(71) Applicant: GREEN CROSS WELLBEING CORPORATION, Seoul (KR)

(72) Inventors: Hae Jung HAN, Yongin-si (KR); Jong Bok YUN, Yongin-si (KR); Joo Young KIM, Yongin-si (KR); Man Heun KIM, Yongin-si (KR); Gu Won JEONG, Yongin-si (KR); Yun Ju LEE, Yongin-si (KR)

(73) Assignee: GREEN CROSS WELLBEING CORPORATION, Seoul (KR)

(21) Appl. No.: 18/593,373

(22) Filed: Mar. 1, 2024

(30) Foreign Application Priority Data
Mar. 2, 2023 (KR) 10-2023-0027520

(51) Int. Cl. A61K 36/287; A61K 9/20; A61K 31/235; A61K 31/7048; A61P 29/00

(52) U.S. Cl. CPC A61K 36/287; A61K 9/2009; A61K 9/2013; A61K 9/2054; A61K 31/235; A61K 31/7048; A61P 29/00; A61K 2236/331; A61K 2236/333; A61K 2236/53

(57) ABSTRACT

A Chrysanthemum zawadskii (abbrev. to “Chrysanthemum”) extract with adjusted contents of each of indicator components of Chrysanthemum zawadskii (abbrev. to “Chrysanthemum”) is disclosed. The indicator components include linarin, chlorogenic acid and isochlorogenic acid. Also disclosed is a method for setting a standard for contents of the indicator components by identifying causes of the difference in properties of the Chrysanthemum extract or raw material and standardizing a preparation process from the analyzed causes of the difference in properties, to obtain a Chrysanthemum extract standardized by setting a content range of each indicator component in the Chrysanthemum raw material. Uses of the extract or a composition containing the extract are disclosed.

### CROSS REFERENCE TO RELATED APPLICATIONS

This application claims priority based on Korean Patent Application No. 10-2023-0027520 filed Mar. 2, 2023, of which entire content is incorporated by reference.

### BACKGROUND OF THE INVENTION

### 1. Field of the Invention

The present invention relates to Chrysanthemum Zawadskii extract and a method for preparation of the same.

### 2. Description of the Related Art

Chrysanthemum Sibiricum is a perennial herb in the Asteraceae family that grows wild at the foot of mountains, riversides, and fields across the country, and is distributed in Korea, northern China, Manchuria, and Siberia. It is known to have hot properties and strong anti-inflammatory and analgesic effects. Therefore, according to herbal remedy and home remedy, the entire Chrysanthemum herb with flowers thereof gathered and dried in the shade has been used as a home medicine for women who have cold hands and feet or a cold body after childbirth, and is also known to be effective in treating hypertension and gastrointestinal diseases.

In the present invention, causes of difference in separate properties of Chrysanthemum Zawadskii extract or original material were identified thus to standardize a manufacturing process thereof, such that indicator components of Chrysanthemum extract having antioxidation or pain-relieving effects, that is, content standards for linarin, chlorogenic and acid isochlorogenic acid A were defined, and a specific method for production thereof with constant properties was developed. Therefore, the present invention has been completed on the basis of the above development.

### PRIOR ART DOCUMENT

### Patent Document

Korean Patent Laid-Open Publication No. 2011-0117788

### SUMMARY OF THE INVENTION

An object of the present invention is to provide a standardized Chrysanthemum Zawadskii (abbrev. to Chrysanthemum) extract and a method for preparation of the same.

Another object of the present invention is to provide a health functional food for antioxidation or for preventing or improving pain, including standardized Chrysanthemum extract.

Further, another object of the present invention is to provide a pharmaceutical composition for antioxidation or for preventing or improving pain, including standardized Chrysanthemum extract.

To achieve the above objects, the following technical solutions are adopted in the present invention.

1. A Chrysanthum extract including 5.5 to 9.9 mg/g of linarin, 1.0 to 2.0 mg/g of chlorogenic acid, and 0.8 to 1.6 mg/g of isochlorogenic acid A.

2. The Chrysanthemum extract according to the above 1, wherein the extract has a particle size of 100 to 150 μm.

3. The Chrysanthemum extract according to the above 1, wherein the extract includes 5.9 to 8.9 mg/g of linarin, 1.2 to 1.7 mg/g of chlorogenic acid, and 1.0 to 1.4 mg/g of isochlorogenic acid A.

4. The Chrysanthemum extract according to the above 1, wherein the extract is prepared by: performing extraction of Chrysanthemum raw material with ethanol at 40 to 60° C. for 5 to 7 hours or hot water extraction of the raw material with hot water at 100 to 120° C. for 2 to 3 hours, or immersing the raw material in ethanol followed by supersonic extraction for 2 to 3 weeks to obtain a supernatant; and drying and pulverizing the supernatant.

5. The Chrysanthemum extract according to the above 4, wherein the Chrysanthemum raw material includes 9.7 to 14.5 mg/g of linarin, 1.3 to 2.5 mg/g of chlorogenic acid, and 1.7 to 3.1 mg/g of isochlorogenic acid A.

6. A method for preparation of the Chrysanthemum extract according to any one of the above 1 to 5, including: performing extraction of Chrysanthemum raw material with ethanol at 40 to 60° C. for 5 to 7 hours or hot water extraction of the raw material with hot water at 100 to 120° C. for 2 to 3 hours, or immersing the raw material in ethanol followed by supersonic extraction for 2 to 3 weeks to obtain a supernatant; and drying and pulverizing the supernatant.

7. The method according to the above 6, wherein the Chrysanthemum raw material includes 9.7 to 14.5 mg/g of linarin, 1.3 to 2.5 mg/g of chlorogenic acid, and 1.7 to 3.1 mg/g of isochlorogenic acid A.

8. The method according to the above 6, further including passing the dried Chrysanthemum extract through a hole having a size of 70 to 90 meshes.

9. A health functional food for antioxidation or for preventing or improving pain, including the Chrysanthemum extract according to any one of the above 1 to 5.

10. A pharmaceutical composition for antioxidation or for preventing or treating pain, including the Chrysanthemum extract according to any one of the above 1 to 5.

Using the method of the present invention, Chrysanthemum extract having constant properties and containing standardized components may be prepared.

The health functional food of the present invention may have constant properties, contain standardized components, and exhibit effects of preventing or treating pain as well as antioxidation.

The pharmaceutical composition of the present invention may have constant properties, contain standardized components, and exhibit effects of preventing or treating pain as well as antioxidation.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is chromatograms showing areas occupied by three types of indicator (CA, components ICA and Linarin), respectively, in regard to four production LOTs of Chrysanthemum extract;

FIGS. 2A to 2F are chromatograms showing areas occupied by three types of indicator components (CA, ICA and Linarin), respectively, in regard to the Chrysanthemum raw materials produced from 2014 till 2020, wherein FIG. 2A is a chromatogram of the Chrysanthemum raw material produced in 2014; likewise, FIGS. 2B, 2C, 2D, 2E and 2F are chromatograms of the Chrysanthemum raw materials produced in 2015, 2016, 2018, 2019 and 2020, respectively;

FIG. 3 shows the types of indicator components;

FIG. 4 illustrates a difference in properties in accordance with the difference in individual meshes;

FIG. 5 illustrates results of confirming the separation of leaves and stems among the raw material by year, and a mixing ratio thereof;

FIG. 6 illustrates a difference in properties before and after extraction following the separation of leaves and stems;

FIG. 7 is a schematic view showing a time-line in a human body application test;

FIG. 8 illustrates results of confirming analgesic efficacy through comparison of reaction times after administration in accordance with the dose of Chrysanthemum extract in a hot plate mouse analgesic model;

FIG. 9 is a schematic view showing a time-line in an acetic acid-derived analgesic efficacy test; and

FIG. 10 is a graph showing results of counting the number of incidence of twist syndrome after administration in accordance with the dose of Chrysanthemum extract.

### DETAILED DESCRIPTION OF THE INVENTION

Hereinafter, the present invention will be described in detail.

The present invention relates to a Chrysanthemum extract including 5.5 to 9.9 mg/g of linarin, 1.0 to 2.0 mg of chlorogenic acid, and 0.8 to 1.6 mg/g of isochlorogenic acid A.

For example, in the extract, the linarin may be included in an amount of 5.9 to 8.9 mg/g or 7 to 8 mg/g, the chlorogenic acid may be included in an amount of 1.2 to 1.7 mg/g or 1.5 to 1.6 mg/g, and the isochlorogenic acid A may be included in an amount of 1.0 to 1.4 mg/g or 1.1 to 1.3 mg/g.

“Gujeolcho” is a resource plant native to Korea and refers to Chrysanthemum Sibiricum in Chrysanthemum family, and may include Chrysanthemum Sibiricum specifically, (“Chrysanthemum”) or similar plants such as Pocheondendranthema, Halladendranthema, Nakdongdendranthema, Rocky dendranthema, etc., but it is not limited thereto. It doesn't matter that any part of Chrysanthemum such as flowers, leaves, stems and roots is used.

In the present invention, the extract means an active ingredient separated from a natural product, is prepared by an extraction process using water, an organic solvent or a mixed solvent thereof, and may include an extract obtained using water, an organic solvent or a mixed solvent thereof, dried powder thereof, or all formulated forms prepared using the same. Further, the extract may also include fractions of the extract that has undergone the extraction process.

The Chrysanthemum extract including 5.5 to 9.9 mg/g of linarin, 1.0 to 2.0 mg/g of chlorogenic acid and 0.8 to 1.6 mg/g of isochlorogenic acid A is substantially equal to the samples used in the experimental groups of Examples 6 to 8 according to the present invention which will be described below, therefore, may have equal antioxidation effect, or pain-preventing, improving or treating effect. Further, a process of preparing Chrysanthemum extract may be standardized on the basis of the above components.

The antioxidation may be, for example, achieved by reduction of thiobarbitric acid reactive substance (TBARS) or matrix metalloproteinase (MMP).

Thiobarbituric acid reactive substance (TBARS) level is for determining improvement of oxidative damage, and is a substance produced by reacting malondialdehyde (MDA), which is generated through peroxidation with a variety of biomembranes, with 2-thiobarbituric acid (TBA). As an indicator relevant to cell damage due to free radicals, it can be determined that the higher the TBARS level, the greater the oxidative damage.

Further, in order to determine the improvement of the oxidative damage, it is possible to confirm the level of matrix metalloproteinase (MMP). When active oxygen caused by the oxidative damage is increased, MMP expression is activated. Therefore, it could be determined that, if the MMP level was decreased, the oxidative damage was improved. Specifically, it may be MMP-3.

In the present invention, antioxidation may be to prevent, improve or treat oxidative damage of synovial cells.

Synovial cells refer to cells of the synovial membrane that covers an inner surface of the joint cavity, and the health functional food of the present invention may prevent, improve or treat oxidative damage to the synovial cells that may occur due to oxidative stress of reactive oxygen species (ROS).

The oxidative damage to the synovial cells may be, for example, oxidative damage to synovial cells in a patient with degenerative arthritis, but it is not limited thereto.

Further, the Chrysanthemum extract may have excellent antioxidation effect, thus to exhibit performance of preventing or improving various diseases related to oxidation. For example, the health functional food of the present invention may exhibit prevention or improvement performance to the disease selected from the group consisting of: Alzheimer's disease, Parkinson's disease, Huntington's disease, amyotrophic lateral sclerosis, multiple sclerosis, obesity, hyperglycemia, dyslipidemia, hypertension, diabetes mellitus, heart failure, cardiovascular ischemia, cerebral ischemic injury, stroke, myocardial infarction, systemic lupus erythematosus, COPD, asthma, acute kidney injury, chronic kidney disease, diabetic nephropathy, end-stage renal disease, viral hepatitis, liver cirrhosis, inflammatory bowel disease, viral infectious disease, solid cancer, hematologic cancer, sepsis, recurrent polychondritis, Kienbock's disease in adults, osteochondrosis of the carpal meniscus, complex regional pain syndrome, Paget's disease of unspecified bone, Paget's disease of other bones, Paget's disease of the skull, progressive ossified fibrodysplasia, relapsing steatosis, multifocal fibrosclerosis, diffuse Fasciitis, polymyalgia rheumatica, Behcet's disease, mixed connective tissue disease, Sjegren's syndrome with myopathy, dry syndrome, Sjegren's syndrome with keratoconjunctivitis, Sjegren's syndrome with lung involvement, Sjegren's syndrome with renal tubule-interstitial disorder, systemic sclerosis with pulmonary involvement, systemic sclerosis with myopathies, Crest's syndrome, combination of calcification-Raynaud's phenomenon-esophageal dysfunction-sclerosis dactylitis-capillary dilatation, progressive systemic sclerosis, polymyositis, other dermatomyositis, juvenile dermatomyositis, systemic lupus erythematosus with organ or systemic involvement, microscopic polyarteritis, aortic arch syndrome, Wegener's granulomatosis, lethal median granuloma, thrombotic thrombocytopeni purpura, thrombotic microangiopathy, Goodpasture's syndrome, juvenile polyarteritis, polyarteritis with pulmonary involvement, polyarteritisnodosa, juvenile polyarthritis, chronic juvenile polyarthritis, systemically onset juvenile arthritis, juvenile rheumatoid spondylitis, juvenile rheumatoid arthritis with or without a rheumatoid factor, juvenile rheumatoid arthritis and adult-onset Still's disease.

Paget's disease may: for example, Paget's disease of unspecified bones, Paget's disease of other bones, or Paget's disease of the skull, but it is not limited thereto.

Sjegren's syndrome may include, for example, Sjegren's syndrome with myopathy, Sjegren's syndrome with keratoconjunctivitis, Sjegren's syndrome with pulmonary involvement, or Sjegren's syndrome with renal tubule-interstitial disorder, but it is not limited thereto.

The systemic sclerosis may include, for example, systemic sclerosis with pulmonary involvement, systemic sclerosis with myopathy, or progressive systemic sclerosis, but it is not limited thereto.

Crest syndrome may include, for example, a combination of calcification, Raynaud's phenomenon, esophageal dysfunction, sclerosis dactylitis and capillary dilatation, but it is not limited thereto.

The dermatomyositis may include, for example, other dermatomyositis or juvenile dermatomyositis, but it is not limited thereto.

The systemic lupus erythematosus may include, for example, systemic lupus erythematosus with organ or systemic involvement, but it is not limited thereto.

The polyarteritis may include, for example, microscopic polyarteritis, juvenile polyarteritis, polyarteritis with pulmonary involvement, or polyarteritisnodosa, but it is not limited thereto.

The polyarthritis may include, for example, juvenile polyarthritis or chronic juvenile polyarthritis, but it is not limited thereto.

The juvenile arthritis may include, for example, systemically onset juvenile arthritis, but it is not limited thereto.

The rheumatoid arthritis may include, for example, juvenile rheumatoid arthritis with or without a rheumatoid factor, or juvenile rheumatoid arthritis, but it is not limited thereto.

Among the diseases listed above, recurrent polychondritis, Kienbock's disease in adults, osteochondrosis of the carpal meniscus, complex regional pain syndrome, Paget's disease of unspecified bone, Paget's disease of other bones, Paget's disease of the skull, progressive ossified fibrodysplasia, relapsing steatosis, multifocal fibrosclerosis, diffuse Fasciitis, polymyalgia rheumatica, Behcet's disease, mixed connective tissue disease, Sjegren's syndrome with myopathy, dry syndrome, Sjegren's syndrome with keratoconjunctivitis, Sjegren's syndrome with lung involvement, Sjegren's syndrome withrenal tubule-interstitial disorder, systemic sclerosis with pulmonary involvement, systemic sclerosis with myopathies, Crest's syndrome, combination of calcification-Raynaud's phenomenon-esophageal dysfunction-sclerosis dactylitis-capillary dilatation, progressive systemic sclerosis, polymyositis, other r dermatomyositis, juvenile dermatomyositis, systemic lupus erythematosus with organ or systemic involvement, microscopic polyarteritis, aortic arch syndrome, Wegener's granulomatosis, lethal median granuloma, thrombotic thrombocytopenia purpura, thrombotic microangiopathy, Goodpasture's syndrome, juvenile polyarteritis, polyarteritis with pulmonary involvement, polyarteritisnodosa, juvenile polyarthritis, chronic juvenile polyarthritis, systemically onset juvenile arthritis, juvenile rheumatoid spondylitis, juvenile rheumatoid arthritis with or without a rheumatoid factor, juvenile rheumatoid arthritis and adult-onset Still's disease may be rare diseases designated by the Korea Centers for Disease Control and Prevention (“KCDC”), and details of such rare diseases are described in rare disease helpline of KCDC, but they are not limited thereto.

The diseases listed above refer to diseases caused by oxidative damage, and in addition to the above listed diseases, diseases that are generally known to be caused by oxidative damage, for example, neurodegenerative diseases, metabolic syndrome, cardiovascular disorders, autoimmune diseases, inflammatory lung disease, kidney disease, liver disease, digestive disease, viral infectious disease, cancer, other inflammatory diseases, or the like may also be included, but they are not limited thereto.

In the present invention, the term ‘pain’ is used as a broad range of meaning that includes dysesthesia derived from stimulation to nerve terminals, and may include nociceptive pain occurring due to tissue damage by a pain causing factor, inflammatory pain related to tissue damage and inflammation, and neuropathic pain caused by functional abnormality of nerve tissues.

For example, the pain may include somatic pain, visceral pain, dysfunctional pain, idiopathic pain, superficial pain, deep pain, itching, migraine and cancer pain, but it is not limited thereto.

The pain may be divided into acute or chronic pain according to duration.

The nociceptive pain may be caused by the damage of skin or tissue located in an internal organ.

Causes of the nociceptive pain may include heat sense, chemical stimulus, physical stimulus, cold sense, or the like, but they are not limited thereto. Further, the nociceptive pain may include pain caused by pierced wound, crush wound, bruise, fracture, burn, etc.

In the present invention, it was confirmed that chlorogenic acid, isochlorogenic acid A, and linarin are indicator components occupying 50% of a sample (No. 71005) within the samples used in antioxidation or pain-relieving experiments. Further, all samples (No. 12001, 12002 and 12003) for proving equality ranges from 80 to 120%, compared to the standard sample (No. 71005), in terms of the chromatogram, peak area of the indicator component and the content range of the indicator component, and it could be confirmed that these samples have equality to one another.

The above properties may include color tone and brightness of Chrysanthemum extract, and further include properties of the Chrysanthemum extract or dried powder thereof. The property of the dried powder may be dull greenish brown (see FIG. 5 of the present invention).

The Chrysanthemum extract may have a particle size of 80 to 150 μm, 80 to 130 μm, 80 to 120 μm, 80 to 110 μm, 100 to 150 μm, 100 to 130 μm, 100 to 120 μm, 100 to 110 μm, preferably, 100 to 150 μm.

The Chrysanthemum extract may be extracted by any method known in the art, for example, may be a hot water extract or an ultrasonic extract.

For example, the extract may be prepared by: performing extraction of Chrysanthemum raw material with ethanol at 40 to 60° C. for 5 to 7 hours or hot water extraction of the raw material with hot water at 100 to 120° C. for 2 to 3 hours, or immersing the raw material in ethanol followed by supersonic extraction for 2 to 3 weeks to obtain a supernatant; and drying and pulverizing the supernatant.

The ethanol used herein may be, for example, 50 to 100% ethanol, 50 to 96% ethanol, 60 to 90% ethanol, 60 to 80% (v/v) ethanol. A solvent used for the ethanol may be water.

The drying may be performed within a range, in which useful components of the extract are not destroyed, by any existing method known in the art. For example, the drying may be performed through lyophilization, drying under reduced pressure, hot air drying, vacuum drying or natural drying.

The pulverization may be performed within a range in which useful components of the extract are not destroyed, by any existing method known in the art, and for example, may be performed using a crusher.

The extract may be prepared, for example, by the preparation method further including passing the dried Chrysanthemum extract through a hole, for example, having a size of 70 to 90 meshes, 75 to 85 meshes, or 79 to 81 meshes. As a result, it is possible to obtain Chrysanthemum extract having uniform particle size and properties. For example, it is possible to obtain the Chrysanthemum extract having a particle size in the range of 80 to 150 μm, 80 to 130 μm, 80 to 120 μm, 100 to 180 μm, 100 to 150 μm, 100 to 130 μm, 100 to 110 μm, preferably 100 to 150 μm. The dried powder of Chrysanthemum extract may have the property of dull greenish brown (see FIG. 5 of the present invention).

The Chrysanthemum raw material may include 9.7 to 14.5 mg/g of linarin, 1.3 to 2.5 mg/g of chlorogenic acid, and 1.7 to 3.1 mg/g of isochlorogenic acid A. The Chrysanthemum raw material may contain the same standard components as those in the standard sample used in the experiments of the present invention, and it is possible to prepare an extract having equality to the standard sample through extraction of the raw material.

Further, the present invention relates to a method for preparation of the Chrysanthemum extract, which includes: performing extraction of Chrysanthemum raw material with ethanol at 40 to 60° C. for 5 to 7 hours or hot water extraction of the raw material with hot water at 100 to 120° C. for 2 to 3 hours, or immersing the raw material in ethanol followed by supersonic extraction for 2 to 3 weeks to obtain a supernatant; and drying and pulverizing the supernatant.

A detailed description of the Chrysanthemum extract is the same as described above.

The ethanol used herein may be, for example, 50 to 100% ethanol, 50 to 96% ethanol, 60 to 90% ethanol, or 60 to 80% ethanol.

The drying may be performed within a range, in which useful components of the extract are not destroyed, by any existing method known in the art. For example, the drying may be performed through lyophilization, drying under reduced pressure, hot air drying, vacuum drying or natural drying.

The pulverization may be performed within a range in which useful components of the extract are not destroyed, by any existing method known in the art, and for example, may be performed using a crusher.

The Chrysanthemum raw material may include 9.7 to 14.5 mg/g of linarin, 1.3 to 2.5 mg/g of chlorogenic acid, and 1.7 to 3.1 mg/g of isochlorogenic acid A.

The Chrysanthemum raw material may contain the same standard components as those in the standard sample used in the experiments of the present invention, and it is possible to prepare an extract having equality to the standard sample through extraction of the raw material.

By extraction of the Chrysanthemum raw material according to the above method, Chrysanthemum extract including 5.5 to 9.9 mg/g of linarin, 1.0 to 2.0 mg/g of chlorogenic acid and 0.8 to 1.6 mg/g of isochlorogenic acid A may be prepared. The prepared extract may exhibit pain-relieving or antioxidation effects as described above, in addition, have equality to the standard sample used in the examples of the present invention.

Accordingly, the above preparation method may be used as a standard process for preparing a substance equal to the above standard sample.

The above preparation method may include the step of passing the dried Chrysanthemum extract through a hole having a size of 70 to 90 meshes. Alternatively, the hole size may range from 75 to 85 meshes, or 79 to 81 meshes.

The hole may have a screen form and may be a structure included in a crusher.

The dried powder passed through the above sized hole may have uniform particle size and properties. The particle size may range from 80 to 150 μm, 80 to 130 μm, 80 to 120 μm, 100 to 180 μm, 100 to 150 μm, 100 to 130 μm, 100 to 110 μm, preferably 100 to 150 μm. The dried powder of Chrysanthemum extract may have the property of dull greenish brown (see FIG. 5 of the present invention).

Further, the present invention relates to a health functional food for antioxidation or for prevention or improvement of pain, which includes the Chrysanthemum extract described above.

Further, the present invention relates to a pharmaceutical composition for antioxidation or for prevention or treatment of pain, which includes the Chrysanthemum extract described above.

Specific details of the Chrysanthemum extract, and pain-relieving or antioxidation effects thereof are the same as described above.

The health functional food or pharmaceutical composition of the present invention may include Chrysanthemum extract granules prepared by mixing the Chrysanthemum extract with a dextrin-based compound.

The dextrin-based compound serves to change properties of the Chrysanthemum extract basically having a high viscosity (e.g., starch syrup-like form) into granular properties, and may be used in an amount of 50 to 70% by weight (“wt. 8”), preferably 55 to 65 wt. %, and more preferably about 60 wt. % based on a total weight of the Chrysanthemum extract granules. Meanwhile, as used herein, the term “granules” may include fine powder, specifically, a mixture of granular particles and fine powder particles, or may be formed by agglomerating fine powder particles. In this case, a size of the granular particles is not particularly limited as long as it can achieve the purpose of the present invention to improve stability such as a particle size of pharmaceuticals generally manufactured in the art.

If a content of the dextrin-based compound is less than 50 wt. % based on the total weight of the Chrysanthemum extract granules, a raw material may be prepared in the form of wet (containing large moisture) granules or powder compared to the existing ones. Therefore, when a finished medicine product is manufactured using the raw material, a binding property of the raw material may be increased to reduce anti-deliquescent effects of a natural extract, thereby delaying a disintegration time. Further, if the dextrin-based compound exceeds 70 wt. % based on the total weight of the Chrysanthemum extract granules, a ratio of the Chrysanthemum extract is relatively decreased, such that a large amount of mixture (Chrysanthemum extract+dextrin-based compound) during a formulation test, and as a result, there are problems that tablet formation is reduced and the disintegration time, etc. is altered during production of the finished medicine.

Such a dextrin-based compound may include, for example, maltodextrin, cyclodextrin, other dextrin-based compounds having properties and features similar or identical to the above materials, and a mixture thereof, and it may be preferable to use maltodextrin in consideration of economical aspect.

The health functional food of the present invention may include general food additives in addition to the above-described Chrysanthemum extract or its granules, and whether or not it is suitable as a food additive may be determined by standards and criteria for corresponding items according to general rules and general test methods in the Food Additives Code approved by the Ministry of Food and Drug Safety, or the like, unless otherwise specified.

Examples of the items listed in the Food Additives Code may include chemical compounds such as ketones, glycine, calcium citrate, nicotinic acid, and cinnamic acid; natural additives such as persimmon pigment, licorice extract, crystalline cellulose, millet pigment, and guar gum; mixed preparations such as sodium L-glutamate preparation, alkali agent added to noodles, preservative agent, tar color agent, etc., but they are not limited thereto.

For example, a health functional food in a tablet form may be formed by granulating a mixture of the extract and an excipient, a binder, a disintegrant and other additives in a conventional manner, followed by compression-molding along with addition of a lubricant, or may be formed by directly compression-molding the mixture. Further, the health functional food in the tablet form may contain a flavoring agent, if necessary.

Among health functional foods in the form of capsules, a hard capsule agent may be prepared by filling a mixture of the extract and additives such as excipients in a conventional hard capsule, while a soft capsule agent may be prepared by filling a mixture of the extract and additives such as excipients in a capsule base such as gelatin. The soft capsule agent may further contain a plasticizer such as glycerin or sorbitol, a colorant, a preservative, and the like, if necessary.

Further, a health functional food in the form of pills may be prepared by molding a mixture of the extract and an excipient, a binder, a disintegrant, etc. according to any existing method known in the art. Further, if necessary, it may be coated with sucrose or other coating agents, or may be surface-coated with a material such as starch, talc, etc.

Further, a health functional food in the form of granules may be prepared by processing a mixture of the extract and an excipient, a binder, a disintegrant, etc. into granules according to any existing method known in the art, and may contain a fragrance agent or a flavoring agent, etc. if necessary.

The health functional food may include, for example, beverages, meat, chocolate, foods, confectionery, pizza, ramen, other noodles, gums, candies, ice cream, alcoholic beverages, vitamin complexes, health supplements and the like.

The health functional food may be orally applied for the purpose of nutritional supplements, and the application form is not particularly limited. For example, in the case of oral administration, the daily intake is preferably 5000 mg or less, more preferably 2000 mg or less, and most preferably 1000 mg or less. When formulated as a capsule or tablet, one (1) tablet may be administered with water once a day.

The pharmaceutical composition of the present invention may further include crystalline cellulose in addition to the above-described Chrysanthemum extract granules (formed of Chrysanthemum extract and dextrin-based compound). The crystalline cellulose is a component that can be used together with a lubricant to be described below to ensure stability against moisture. Therefore, when medicating the Chrysanthemum extract according to the present invention, the tablet coating corresponds to a general coating rather than a moisture-proof coating. The crystalline cellulose may be used in an amount of 50 to 70 wt. %, preferably 50 to 65 wt. %, and more preferably 50 wt. % or more and less than 60 wt. % based on the total weight of the pharmaceutical composition of the present invention. If a total weight of an initial finished composition is rather high when considering a size of tablets, a content or weight of the crystalline cellulose and a lubricant described below may be reduced. That is, a ratio of additives may be adjusted in consideration of the tablet size.

If the content of the crystalline cellulose is less than 50 wt. % based on the total weight of the pharmaceutical composition of the present invention, the disintegration time may be delayed compared to the existing composition. Further, anti-deliquescent effects may also be decreased, such that a change in stability due to the increased water content may occur. Further, when the content of the crystalline cellulose exceeds 70 wt. % based on the total weight of the pharmaceutical composition of the present invention, a ratio of the active ingredient in the mixture of the same weight is relatively reduced due to an increase in the content of the crystalline cellulose, and a tablet size may vary because tablets should be made with an excessive amount of mixture. Further, when maintaining the tablet size in this state, it is necessary to increase a tablet-punching pressure, which may affect disintegration due to an increase in the hardness. Furthermore, when tablet-punching, the surface is not smooth and a binding force is reduced, which in turn increases friability and may eventually cause a problem in tablet-punching ability and coating.

The pharmaceutical composition with improved stability, which includes the natural extract according to the present invention, may further include a pharmaceutically acceptable excipient, but does not include a solubilizer and a disintegrant.

The excipient may be a lubricant, and the lubricant may include any lubricant commonly used in the art, for example, silicon dioxide, magnesium stearate, and a mixture thereof, but it is preferable to apply silicon dioxide and magnesium stearate together. Further, the lubricant may be used in an amount of 2 to 4 wt. % based on the total weight of the pharmaceutical composition of the present invention.

On the other hand, the pharmaceutical composition with improved stability, which includes the natural extract according to the present invention, may be formulated in the form selected from the group consisting of tablets, pills, powders, granules, capsules, suspensions, solutions, emulsions, syrups, and lyophilizing preparations. However, it is most preferable to formulate in the form of a tablet in consideration of the spirit of the present invention.

When the pharmaceutical composition is formulated in the form of a tablet, the tablet may include a coating layer, and the coating layer may include only an aqueous coating agent. That is, as described above, as the present invention secures stability against moisture by applying a specific amount of the crystalline cellulose and the lubricant, etc., the existing moisture-proof coating agent is not included (that is, the pharmaceutical composition may exhibit moisture-proof effects even without a moisture-proof coating agent, and such moisture-proof effects may be expressed by any one or more among a dextrin-based compound, crystalline cellulose and a lubricant).

The water-based coating agent may include, for example, hydroxypropylmethyl cellulose (HPMC), glycerin fatty acid ester, titanium dioxide, Lac pigment, and substances having properties and features similar or identical to the above materials. At this time, the hydroxypropylmethyl cellulose can be replaced with methyl cellulose, ethyl cellulose, hydroxypropyl cellulose, or a cellulosic compound having properties similar to the above materials. In addition, the water-based coating agent may further include any one or more of a solubilizer, a plasticizer, a binder, a lubricant, a wetting agent, a sweetener, a flavoring agent, an emulsifier, a suspending agent, and a diluent, if necessary.

The pharmaceutical composition of the present invention, which includes the above components, has various benefits in addition to the advantages described above. First, the pharmaceutical composition of the present invention may reduce the content of the drug since not using a solubilizer, a disintegrant, and a moisture-proof coating agent unlike the conventional ones. Further, in the case of a general tablet, the hardness is increased as the water content is decreased, such that the disintegration time also tends to be increased. However, in the case of a tablet formulated using the pharmaceutical composition of the present invention, the hardness is not increased as the moisture content is maintained, therefore, has an advantage that a time required for disintegration is constant within 30 minutes even without the use of a disintegrant (not affected by storage temperature and period).

### EXAMPLES

### 1. Preparation of Chrysanthemum Extract

Chrysanthemum extract may be prepared by organic solvent extraction, hot water extraction, and organic solvent extraction and supersonic extraction, but they are not limited thereto.

### 1-1) Organic Solvent Extraction Method

To 50 kg of Chrysanthemum raw material, 70% ethanol was added in an amount of 20 times of the raw material, followed by extraction at 50° C. for 5 to 8 hours. Then, the product was filtered through 5 μm filter, concentrated at 50° C. to reach Brix sweetness of 60±10, and mixed with maltodextrin in a mixing ratio of raw material:maltodextrin of 6:4, 5:5 and 4:6, respectively. Thereafter, the product was dried under reduced pressure at 50° C. for 16 to 20 hours and then passed through a screen of 80 meshes.

### 1-2) Hot Water Extraction Method

In order to prepare a hot water extract of Chrysanthemum Sibiricum after washing this natural herb with distilled water and finely pulverizing it with a crusher, the pulverized product was heated in a boiling water to obtain the Chrysanthemum extract. At this time, after adding 1.0 L of water to each 100 g of Chrysanthemum raw material, it was extracted by heating in the boiling water at 100 to 120° C. for about 2 to 3 hours, and a cooler was installed to maintain a constant temperature and prevent loss by steam. The obtained Chrysanthemum extract was filtered to obtain a supernatant, followed by concentration using a concentrator. The Chrysanthemum extract powder was prepared by freezing the Chrysanthemum concentrate and lyophilizing the same at −80° C. As the drying method, in addition to the lyophilizing, vacuum drying or hot air drying may be used, but it is not limited to the above method.

### 1-3) Organic Solvent and Ultrasonic Extraction Method

In order to prepare an organic solvent extract and ultrasonic extract of Chrysanthemum Sibiricum, this natural herb was immersed in 70% ethanol and extracted for about 2 to 3 weeks by an ultrasonic extractor to obtain an extract, then only the supernatant was separated and filtered through gauze to remove foreign substances. The alcohol extract obtained by the above process was concentrated using a concentrator and frozen, followed by lyophilizing at −80° C. to prepare an extract powder of the Chrysanthemum extract. In this method, the ultrasonic extractor is used for the purpose of rapid extraction within a short time, but the ultrasonic extraction step may not necessarily be used in mass production. As in the hot water extraction method, vacuum drying or hot air drying may be used instead of lyophilization.

### 2. Determination of Indicator Component

In the present invention, it was intended to measure contents of the following three indicator components. Hereinafter, structures of the following three components and a motive of selecting the same will be described in detail below.

In the present invention, the sample No. 71005 used in the following human body application test was set as a standard sample and, in order to prove equality compared to the above sample, three types of sample production batches (12001, 12002 and 12003) were subjected to analysis of content of an indicator component contained in each of the batches. For each sample, analysis was repeatedly performed three times thus to confirm the results.

In the present experiment, HPLC was performed using Agilent 1260 instrument and chromatogram conditions are as follows:

TABLE 1
Test conditions for HPLC chromatogram

| Division | Chromatogram condition | | |
|---|---|---|---|
| UV wavelength | 330 nm | | |
| Injected amount | 10 mL | | |
| Column temperature | 25° C. | | |
| Temperature of | 10° C. | | |
| sample room | | | |
| Flow rate | 1.0 mL/min | | |
| | Minutes | Solution A (%) | Solution B (%) |
| | (min) | 0.025M KH₂PO₄ | 100% acetonitrile |
| Mobile phase | 0 | 85 | 15 |
| | 12 | 75 | 25 |
| | 20 | 59 | 41 |
| | 30 | 10 | 90 |
| | 30.1 | 85 | 15 |
| | 35 | 85 | 15 |

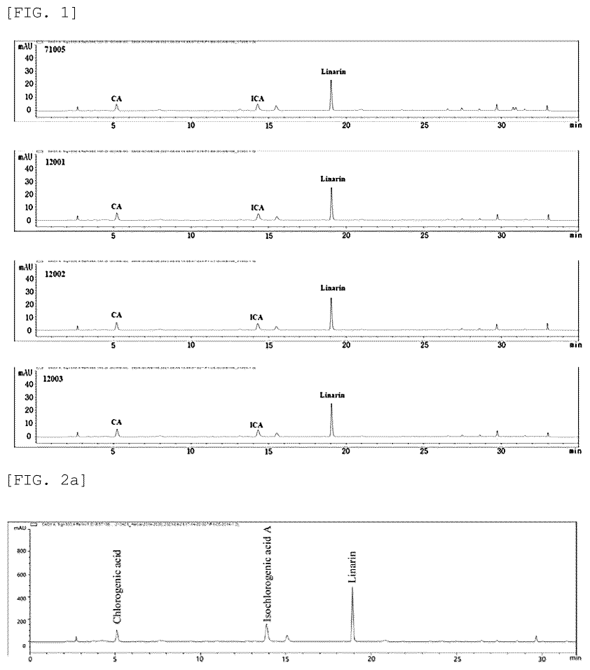

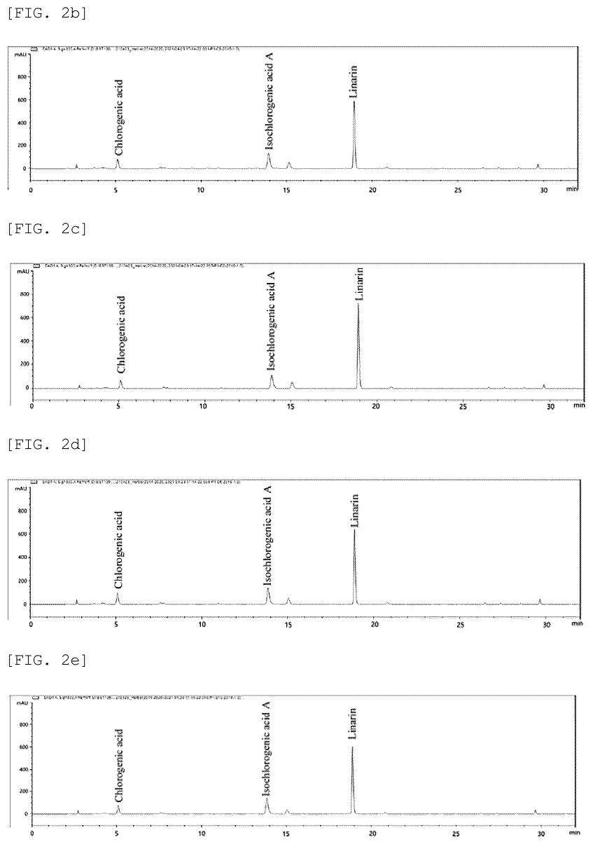

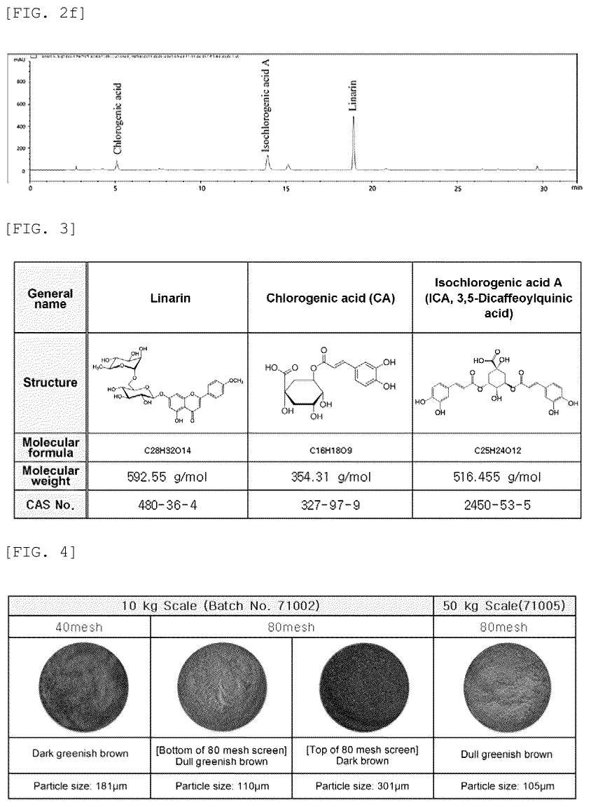

At first, the indicator components (chlorogenic acid, isochlorogenic acid A, and linarin) occupying 50% or more of a peak area in the standard sample (71005) were selected and analyzed in terms of chemical similarity. As a result of the analysis, it was confirmed that the area (%) occupied by the indicator component in the standard sample is 51.94% while the area (%) occupied by the indicator component in each of three equality-proved samples 3 Lots (12001, 12002 and 12003) is 59, 07% such that the indicator component is 50% or more in all samples (FIG. 1). From the results, it was confirmed that three indicator components (chlorogenic acid, isochlorogenic acid A, and linarin) in the Chrysanthemum extract are good as the indicator component capable of proving equality to the standard sample.

### 3. Proving of Equality Between Samples

Between the standard sample and the sample requiring proof of equality, the area occupied by three indicator components in the chromatogram was compared. As a result of the comparison, it was confirmed that when the area of each component in the standard sample is assumed to 100%, the area of each indicator component is included within the range of 80 to 120% in all of three samples, as compared to the standard sample.

TABLE 2
Area of indicator component in chromatogram
of Chrysanthemum extract

| | | Standard | | | |
|---|---|---|---|---|---|
| Area of indicator | | sample | Equality-proved sample | | |
| component | | (71005) | 12001 | 12002 | 12003 |
| CA | 34.2~51.2 | 42.7 | 50.0 | 49.1 | 51.2 |
| ICA | 42.6~63.8 | 53.2 | 58.9 | 54.9 | 59.6 |
| Linarin | 121.8~182.8 | 152.3 | 164.7 | 164.1 | 168.6 |

TABLE 3
Content of indicator component in Chrysanthemum extract (mg/g)

| Content of indicator | | Standard | | | |
|---|---|---|---|---|---|
| component | | sample | Equality-proved sample | | |
| (71005) | | (71005) | 12001 | 12002 | 12003 |
| CA | 1.2~1.7 | 1.4 | 1.6 | 1.6 | 1.7 |
| ICA | 1.0~1.4 | 1.2 | 1.2 | 1.2 | 1.2 |
| Linarin | 5.9~8.9 | 7.4 | 7.8 | 7.8 | 8.0 |
| External | 6.38~9.57 | 7.62 | 7.94 | 7.88 | 8.11 |
| experiment | | | | | |
| Linarin | | | | | |

As shown in Tables 3 and 4 above, with regard to the chromatogram, the peak area of indicator component and the content range of indicator component between sample 3 batches requiring proof of equality to the standard sample, all values are within 80 to 120% compared to the standard sample thus to obtain a result, from which appropriate decision could be made. Therefore, it was demonstrated that all of the samples requiring the proof of equality are substantially equal to the standard sample, and it was confirmed that the preparation process of the present invention has been standardized.

### 4. Identification of Difference in Properties

In the following experiments, it was intended to identify the cause of a difference in properties between the samples.

### 4-1) Effect of Production Scale

With regard to study samples (71001-3, 71007-9 and small scale), a standard and conditions of an apparatus (concentrator) at the time of production among production facilities, which is expected to influence properties, were investigated to determine whether the above results have an effect on the final extract.

TABLE 4
Difference in properties due to the difference in concentrators

| Amount of | | | |
|---|---|---|---|
| raw material | Batch No. | Property | Concentrator |
| 10 kg | 71001, 71002, | Dark greenish | A |
| | 71003 | brown | |
| 10 kg | 71007, 71008, | Bright greenish | A |
| | 71009 | brown | |
| 50 kg | 71004, 71005, | Dull greenish | B |
| | 71006 | brown | |

In the case of Batch Nos. 71001 to 3 and 71007 to 9 for relatively small scale production as the production facility, the concentrator supplies steam with 1 to 4 kgf/cm² at a high temperature of 110 to 140° C. during steam treatment under reduced pressure. In the above process, it was confirmed that a mailard reaction (“browning”) of the material is progressed. The above apparatus is a research equipment which does not belong to the subjects for GMP facility management, therefore, was confirmed not to cause a significant problem. Since a large scale production sample should be produced in GMP production facility, it was determined that the above problem would not occur in mass production process.

### 4-2) Effect of Crusher Screen Mesh Size

In order to determine the causes influencing the difference in properties according to production process, two types of meshes having a size of 40 meshes and 80 meshes, respectively, were used in the final pulverizing step after drying and investigated whether to cause the difference in properties. As a result, the difference in properties due to a difference in meshes is shown in FIG. 4.

As a result of the experiment, it was clearly confirmed that the difference in properties occurs using 40 meshes and 80 meshes, respectively. It was confirmed that a particle size of the dried powder of the extract influences the brightness and even whole color in the properties as the particle size based on mesh size varies. From the above result, it was confirmed that using a 80 mesh crusher is optimally preferred in a process of standardizing the production method.

### 4-3) Confirmation of Effect of Leaf/Stem Mixing Ratio of Raw Material

### (1) Proportion by Package Unit

In the present step, it was intended to confirm whether the properties are different according to a mixing ratio of Chrysanthemum leaves and stems. In order to investigate uniformed package standards by package unit of the Chrysanthemum raw material supplied from farm, leaves and stems were randomly taken from the raw material under storage (package unit: 25 kg), and about 300 g of the taken leaves and stems was weighed on the scales, followed by determining a mixing ratio of leaves and stems thereof. The measured 300 g of raw materials were separated into leaves and stems, which in turn were placed in 2 L beaker, followed by visibly measuring a volume thereof. As a result of the measurement, the following results shown in FIG. 5 were confirmed.

As a result of investigation based on the raw material sample by year, the mixing ratio of leaves and stems of the Chrysanthemum raw material was 1:2 by volume, confirming that the leaves and stems are constantly mixed. Therefore, it was confirmed that the mixing ratio of leaves and stems does not influence the properties of the raw material.

### 4-4) Difference in Property During Extraction

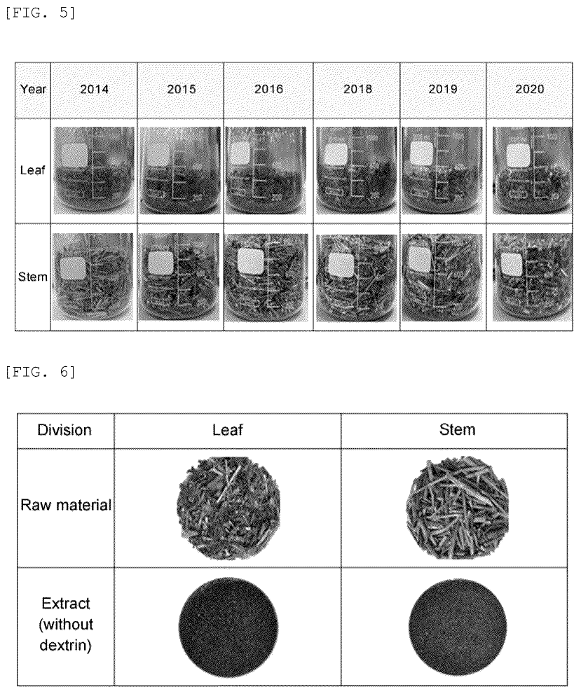

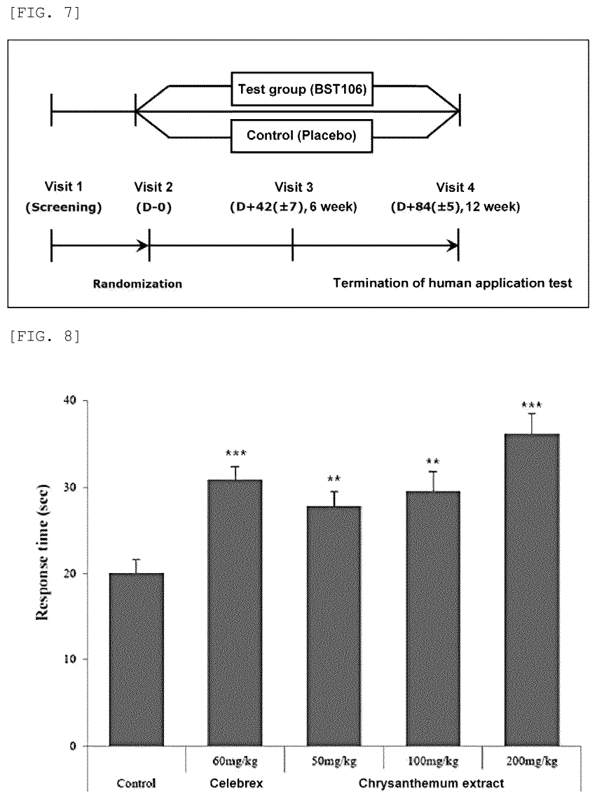

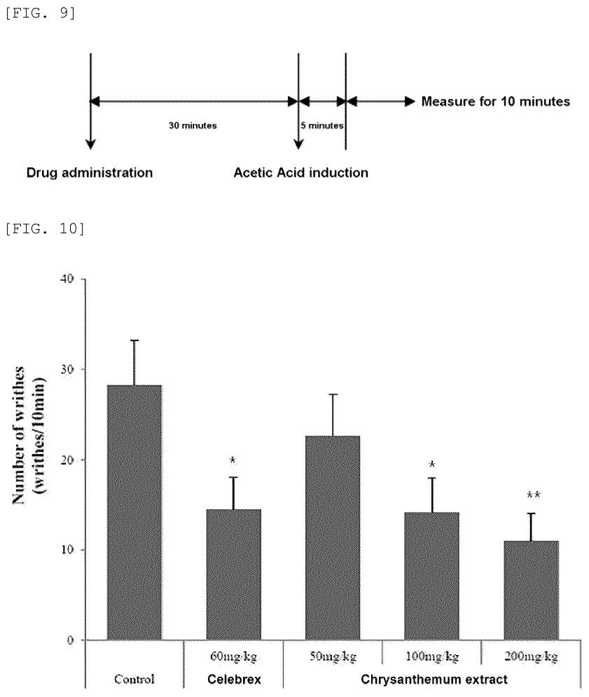

In the present experiment, in order to determine whether the properties are different due to the mixing ratio of leaves and stems during mass production, the leaves and stems were separated using bellows and extracted, followed by comparison of the properties. In the raw material, the difference in properties was visibly confirmed between the leaves and the stems. However, after extraction, the difference in properties was not significant by visual inspection, and thus not distinguishable. The experimental results are shown in FIG. 6.

### 5. Measurement of Content of Indicator Component

In the present experiment, it was intended to select an indicator component among the Chrysanthemum raw materials and to set a content range of the selected indicator component. The indicator components in the Chrysanthemum extract set in the previous step were chlorogenic acid (CA), isochlorogenic acid A (ICA), and linarin. Therefore, it was also intended to investigate the same indicator components in the raw material. As analysis samples, the Chrysanthemum raw materials in 2014 to 2020 were analyzed. The chromatogram was conducted under the conditions shown in Table 1 above. As a result of confirming validity of the indicator components of the raw materials supplied from the farm by year, it was confirmed that standard indicator components (chlorogenic acid, isochlorogenic acid A, and linarin) occupying 50% or more in the Chrysanthemum raw material are maintained in predetermined proportions among the samples, therefore, it was determined that the above three indicator components are also valid as indicator components in the Chrysanthemum raw material.

As a result of analyzing the contents of the indicator components (chlorogenic acid, isochlorogenic acid A, and linarin) of Chrysanthemum raw materials harvested from 2014 to 2020, it was analyzed that chlorogenic acid and isochlorogenic acid A have each content in the range of 70 to 130% of the average value, while linarin has a content in the range of 80 to 120% of the average value. Therefore, such ranges as described above were set to the standards for indicator component.

TABLE 5
Content of indicator component in Chrysanthemum raw material

| mg/g | 2014 | 2015 | 2016 | 2018 | 2019 | 2020 | Average |
|---|---|---|---|---|---|---|---|
| Chlorogenic | 2.2 | 1.9 | 1.5 | 2.1 | 1.6 | 1.8 | 1.9 |
| acid | | | | | | | |
| Isochlorogenic | 2.9 | 2.4 | 1.9 | 2.5 | 2.5 | 2.4 | 2.4 |
| acid A | | | | | | | |
| Linarin | 10.0 | 12.1 | 14.5 | 13.1 | 12.6 | 10.0 | 12.1 |

Based on the result shown in Table 5, linarin as one of the Chrysanthemum raw materials was set to 9.7 to 14.5 mg/g as a standard content; likewise, chlorogenic acid was set to 1.3 to 2.5 mg/g; and isochlorogenic acid A was set to 1.7 to 3.1 mg/g as standard contents thereof.

### 6. Human Application Test and Results of Ingestion of Tablets Containing Chrysanthemum Extract

### 6-1) Human Application Test Procedure

(1) Subjects of the human application test are randomly assigned to a test group or a control only for those who are suitable for human application test after determining whether the selection or exclusion criteria are met through a visit evaluation for people with mild degenerative knee arthritis symptoms. Then, the food for human application test (test food or control food) was supplied for ingestion for 12 weeks. The outline of the human application test is shown in FIG. 7.

(2) The constitutional composition of the test food is shown in Table 6 below, while that of the control food is shown in Table 7 below. In Table 6, the Chrysanthemum extract used herein was an ultrasonic extract prepared by the organic solvent extraction method described in the above preparative example.

TABLE 6

| | Mixing ratio | Content |
|---|---|---|
| Name of raw material | (%) | (mg) |
| Chrysanthemum extract | 41.67 | 250.00 |
| Crystalline cellulose | 52.33 | 314.00 |
| Silicon dioxide | 1.80 | 10.80 |
| Magnesium stearate | 1.20 | 7.20 |
| HPMC | 2.07 | 12.41 |
| Glycerin fatty acid ester | 0.62 | 3.73 |
| Titanium dioxide | 0.21 | 1.24 |
| Lac pigment | 0.10 | 0.62 |
| Sum | 100 | 600 |

TABLE 7

| | Mixing ratio | Content |
|---|---|---|
| Name of raw material | (%) | (mg) |
| Crystalline cellulose | 74.00 | 444.00 |
| Malto-dextrin | 22.00 | 132.00 |
| Magnesium stearate | 0.50 | 3.00 |
| Silicon dioxide | 0.50 | 3.00 |
| HPMC | 2.07 | 12.41 |
| Glycerin fatty acid ester | 0.62 | 3.73 |
| Titanium dioxide | 0.21 | 1.24 |
| Lac pigment | 0.10 | 0.62 |
| Sum | 100 | 600 |

(3) Before entering the human application test, past medical history and drug administration history were checked through past medical records and interviews.

(4) Each of the test group and the control ingested test food (250 mg/day as Chrysanthemum extract) and control food once a day for 12 weeks (84 days).

### 6-2) Experimental Results

### (1) Comparison of TEARS Levels

TABLE 8

| | | Test group | | Control |
|---|---|---|---|---|
| | n | (N = 53) | n | (N = 57) |
| Standard (visit 1) | 53 | 9.16 | 57 | 9.18 |
| 12 week (visit 4) | 53 | 8.95 | 57 | 9.46 |

As shown in Table 8 above, it was demonstrated in the results of analyzing a change amount, i.e., variation of TBARS that the level thereof was decreased by 0.21 UM in the test group, whereas it was increased by 0.26 UM in the control after 12 weeks from ingestion.

In turn, it could be seen that the oxidative damage of synovial cells was improved in the test group in which the TBARS level was decreased, whereas the oxidative damage of the synovial cells was not improved in the control in which the TBARS level was increased.

### (2) Measurement of MMP-3 Levels

Table 9 shows the results of analyzing the variation of MMP-3 by the PP set, and it was confirmed that after 12 weeks from ingestion, the level thereof was decreased by 0.76 ng/ml in the test group, whereas it was increased by 1.20 ng/ml in the control.

TABLE 9

| | |
|---|---|
| Test group | Control |
| N = 53 | N = 57 |

| | n | Mean | n | Mean |
|---|---|---|---|---|
| Standard (visit 1) | 53 | 18.75 | 57 | 18.03 |
| 12 week (visit 2) | 53 | 17.99 | 57 | 19.22 |
| Variation compared | 53 | −0.76 | 57 | 1.20 |
| to standard | | | | |

Further, Table 10 shows results of analyzing the variation of MMP-3 by the FA set, and it was confirmed that after 12 weeks from ingestion, the level thereof was decreased by 0.34 ng/ml in the test group, whereas it was increased by 1.19 ng/ml in the control.

TABLE 10

| | |
|---|---|
| Test group | Control |
| N = 58 | N = 58 |

| | n | Mean | n | Mean |
|---|---|---|---|---|
| Standard (visit 1) | 58 | 18.30 | 58 | 18.03 |
| 12 week (visit 2) | 57 | 17.96 | 58 | 19.22 |
| Variation compared | 57 | −0.42 | 58 | 1.19 |
| to standard | | | | |

Since MMP-3 was decreased in the test group, whereas MMP-3 was increased in the control, it could be determined that the oxidative damage was improved in the test group ingesting the test food (including Chrysanthemum extract).

### 7. Assessment of Analgesic Efficacy of Chrysanthemum Extract (Chrysanthemum 70% Ethanol Extract)

### 7-1) Experimental Method

### (1) Standard

The experimental method used in this example is an analgesic efficacy experiment method using a hot plate test described in the “Guidelines for efficacy test of herbal preparations—General efficacy tests for painful diseases” announced by the Ministry of Food and Drug Safety, wherein the analgesic effect in the central nervous system of a mouse is measured with a response time to a heat stimulus as an index through a hot plate. At this time, the response time to the heat stimulus is measured as a time elapsed until the experimental animal licks the paw or jumps.

### (2) Drug Administration

1) Celebrex (Celecoxib, Pfizer, 200 mg), Saline (Chungwae Pharmaceutical), Chrysanthemum extract (Green Cross HS) and male ICR mice (6 weeks old, weight 30-35 g, Orient Bio) were prepared. In this example, the Chrysanthemum extract was extracted by the organic solvent extraction method described in the above preparative example wherein it was extracted using 70% ethanol.

2) 60 mg/kg of Celebrex and 50, 100 and 200 mg/kg of Chrysanthemum extracts were prepared and orally administered to ICR mice, respectively. At this time, Celebrex is converted into administration concentration and suspended in saline while vortexing, and Chrysanthemum extract is suspended in 0.5% CMC and sonicated at 20 to 25° C. for about 10 minutes.

### 2. Experiment Assessment and Results

### (1) Evaluation Method

According to the efficacy experimental method using the hot plate test method, as the experimental animals, 5-week-old male ICR mice were purchased, acclimatized for 1 week, and then tested. The hot plate was maintained at 55° C., and drug administration was performed 30 minutes before this method. The central pain response due to heat was measured with an index, i.e., the time elapsed until the mouse licks the hind paw or jumps starting from contacting with the hot plate.

(2) Classification of Test Groups (n=8)

TABLE 11

| | |
|---|---|
| 1 | Control (vehicle) |
| 2 | Celebrex 60 mg/kg, oral |
| 3 | Chrysanthemum extract 50 mg/kg, oral |
| 4 | Chrysanthemum extract 100 mg/kg, oral |
| 5 | Chrysanthemum extract 200 mg/kg, oral |

### (3) Experimental Results

After administering the Chrysanthemum extract by concentration as shown in Table 11, and after 20 minutes, the mouse was placed on a hot plate maintained at 55° C. Then, the pain response due to heat was measured with an index, i.e., the time elapsed until the mouse licks the hind paw or jumps starting from contacting with the hot plate. Measured results are shown in FIG. 8 and Table 12.

TABLE 12

| | | | G3 | G4 | G5 |
|---|---|---|---|---|---|
| | | | Chrysan- | Chrysan- | Chrysan- |
| | | G2 | themum | themum | themum |
| Response | G1 | Celecoxib | extract | extract | extract |
| time (sec) | Control | 60 mg/kg | 50 mg/kg | 100 mg/kg | 200 mg/kg |
| 1 | 24.44 | 32.71 | 30.41 | 25.33 | 28.06 |
| 2 | 18.20 | 25.61 | 35.08 | 29.46 | 35.12 |
| 3 | 15.81 | 26.94 | 22.16 | 22.12 | 30.78 |
| 4 | 18.52 | 27.82 | 26.14 | 38.12 | 34.30 |
| 5 | 24.51 | 35.55 | 31.69 | 28.08 | 41.17 |
| 6 | 24.71 | 36.22 | 20.86 | 32.95 | 38.92 |
| 7 | 13.42 | 28.54 | 28.74 | 22.56 | 32.85 |
| 8 | 20.92 | 33.71 | 27.21 | 37.80 | 48.24 |
| Mean | 20.07 | 30.89 | 27.79 | 29.55 | 36.13 |
| % of control | | 53.9 | 38.4 | 47.2 | 80.3 |

In the case of the control, the response time was 20.1 seconds. For the experimental groups administered with 50, 100, and 200 mg/kg of Chrysanthemum extracts, the response time was increased significantly compared to the control, and the increase rates were confirmed to be 38.4%, 47.2%, and 80.3%, respectively. The Celebrex (including celecoxib) administration group as a positive control drug also showed an increase in the hot plate response time to 53.9%.

In turn, the response time was significantly increased in all dose groups to which the Chrysanthemum extract was administered, as compared to the control. In particular, the 200 mg/kg Chrysanthemum administration group had a greater response time compared to the Celebrex administration group. From these results, it could be seen that Chrysanthemum extract can relieve pain in patients by exhibiting a strong central nervous system writhing inhibitory activity.

### 8. Assessment of Analgesic Efficacy for Chrysanthemum Extract (Chrysanthemum 70% Ethanol Extract) in Acetic Acid-Induced Writhing Test Model

### 8-1) Experimental Method

### (1) Standard

The experimental method used in this example is the “Acetic acid writhing test” described in the “Guidelines for efficacy test of herbal preparations—General efficacy tests for painful diseases” announced by the Ministry of Food and Drug Safety, wherein acetic acid is administered intraperitoneally to cause damage of capillaries and pain in the abdominal cavity, and the analgesic effect in peripheral nerves is measured using the abdominal contractile response as an index.

### (2) Drug Administration

1) Celebrex (Celecoxib, Pfizer, 200 mg), acetic acid (Daejunghwageum), Saline (Chungwae Pharmaceutical), Chrysanthemum extract (Green Cross HS) and male ICR mice (5 weeks old, Orient Bio) were prepared. In this example, the Chrysanthemum extract was extracted by the organic solvent extraction method described in the above preparative example wherein it was extracted using 70% ethanol.

2) 60 mg/kg of Celebrex and 50, 100 and 200 mg/kg of Chrysanthemum extracts were prepared and orally administered to ICR mice. At this time, Celebrex is converted into administration concentration and suspended in saline while vortexing, and Chrysanthemum extract is suspended in 0.5% CMC and sonicated at 20 to 25° C. for about 10 minutes.

### 8-2) Assessment and Results

### (1) Evaluation Method

For the experimental method for analgesic effects to the acetic acid writhing test, as the experimental animals, 4-week-old male ICR mice were purchased, acclimatized for 1 week, and then tested. The Chrysanthemum extract and control drug, respectively, were orally administered to mice by dose, followed by intraperitoneal injection of acetic acid (0.7% acetic acid-physiological saline (0.1 ml/10 g B.W.)) 30 minutes after administration. Subsequently, from 5 minutes after injection, the number of occurrences of writhing was measured for 10 minutes (FIG. 3).

(2) Classification of Test Groups (n=6)

TABLE 13

| | |
|---|---|
| 1 | Control |
| 2 | Celebrex 60 mg/kg, oral |
| 3 | Chrysanthemum 50 mg/kg, oral |
| 4 | Chrysanthemum 100 mg/kg, oral |
| 5 | Chrysanthemum 200 mg/kg, oral |

### (3) Experimental Results

The Chrysanthemum extract was administered by concentration as shown in Table 13 and, after 30 minutes, 0.7% acetic acid-saline was administered intraperitoneally to induce pain. Subsequently, from 5 minutes after the intraperitoneal administration, the number of occurrences of writhing syndrome was counted for 10 minutes and results thereof are shown in FIG. 10 and Table 14.

TABLE 14

| | | | G3 | G4 | G5 |
|---|---|---|---|---|---|
| | | | Chrysan- | Chrysan- | Chrysan- |
| Writhing | | G2 | themum | themum | themum |
| syndrome/ | G1 | Celecoxib | extract | extract | extract |
| 10 min | Control | 60 mg/kg | 50 mg/kg | 100 mg/kg | 200 mg/kg |
| 1 | 23.00 | 25.00 | 19.00 | 9.00 | 3.00 |
| 2 | 24.00 | 18.00 | 17.00 | 3.00 | 6.00 |
| 3 | 27.00 | 9.00 | 27.00 | 20.00 | 12.00 |
| 4 | 28.00 | 6.00 | 22.00 | 27.00 | 23.00 |
| 5 | 40.00 | 15.00 | 29.00 | 12.00 | 11.00 |
| 6 | 28.00 | 14.00 | 22.00 | 14.00 | 11.00 |
| Mean | 28.33 | 14.50 | 22.67 | 14.17 | 11.00 |
| % of control | | 48.9 | 20.2 | 50.1 | 61.3 |

In the case of the control, the number of occurrences of writhing syndrome was 28.33/10 min, while the group administered with 100 and 200 mg/kg Chrysanthemum extracts, excluding the group administered with a low dose of 50 mg/kg of Chrysanthemum extract, had the number of occurrences of writhing syndrome reduced by 50.1% and 61.3%, respectively, compared to that of the control. The group administered with Celebrex (including celecoxib) as a positive control also had the number of acetic acid-induced writhing syndromes significantly reduced, and the inhibition rate was confirmed to be 48.9%.

From the above results, it was confirmed that the number of occurrences of writhing syndrome was significantly reduced in all dose groups administered with the Chrysanthemum extract compared to the control, and the number of occurrences of writhing syndrome was further significantly reduced in the 100 mg/kg and 200 mg/kg Chrysanthemum extract administration groups compared to the Celebrex administration group. Therefore, it could be determined that the Chrysanthemum extract can relieve the pain of the patient by exhibiting a strong peripheral nervous system pain inhibitory activity.

What is claimed is:

1. A Chrysanthum extract comprising 5.5 to 9.9 mg/g of linarin, 1.0 to 2.0 mg/g of chlorogenic acid, and 0.8 to 1.6 mg/g of isochlorogenic acid A.

2. The Chrysanthemum extract according to claim 1, wherein the extract has a particle size of 100 to 150 μm.

3. The Chrysanthemum extract according to claim 1, wherein the extract comprises 5.9 to 8.9 mg/g of linarin, 1.2 to 1.7 mg/g of chlorogenic acid, and 1.0 to 1.4 mg/g of isochlorogenic acid A.

4. The Chrysanthemum extract according to claim 1, wherein the extract is prepared by: performing extraction of Chrysanthemum raw material with ethanol at 40 to 60° C. for 5 to 7 hours or hot water extraction of the raw material with hot water at 100 to 120° C. for 2 to 3 hours, or immersing the raw material in ethanol followed by supersonic extraction for 2 to 3 weeks to obtain a supernatant; and drying and pulverizing the supernatant.

5. The Chrysanthemum extract according to claim 4, wherein the Chrysanthemum raw material comprises 9.7 to 14.5 mg/g of linarin, 1.3 to 2.5 mg/g of chlorogenic acid, and 1.7 to 3.1 mg/g of isochlorogenic acid A.

6. A method for preparation of the Chrysanthemum extract according to claim 1, comprising: performing extraction of Chrysanthemum raw material with ethanol at 40 to 60° C. for 5 to 7 hours or hot water extraction of the raw material with hot water at 100 to 120° C. for 2 to 3 hours, or immersing the raw material in ethanol followed by supersonic extraction for 2 to 3 weeks to obtain a supernatant; and drying and pulverizing the supernatant.

7. The method according to claim 6, wherein the Chrysanthemum raw material comprises 9.7 to 14.5 mg/g of linarin, 1.3 to 2.5 mg/g of chlorogenic acid, and 1.7 to 3.1 mg/g of isochlorogenic acid A.

8. The method according to claim 6, further comprising passing the dried Chrysanthemum extract through a hole having a size of 70 to 90 meshes.

9. A health functional food for antioxidation or for preventing or improving pain, comprising the Chrysanthemum extract according to claim 1.

10. A pharmaceutical composition for antioxidation or for preventing or treating pain, comprising the Chrysanthemum extract according to claim 1.

11. A method for preventing or improving pain in a subject, comprising administering an effective amount of a composition comprising the Chrysanthemum extract according to claim 1 to the subject.

12. The method according to claim 11, wherein the composition is a food composition or a pharmaceutical composition.

13. A method for preventing or improving oxidative stress in a subject, comprising administering an effective amount of a composition comprising the Chrysanthemum extract according to claim 1 to the subject.

14. The method according to claim 13, wherein the composition is a food composition or a pharmaceutical composition.
